(19) United States
(12) Patent Application Publication
(10) Pub. No.: US 2002/0090318 A1
(43) Pub. Date: Jul. 11, 2002

(54) Method and device for liberating a fragrant or disinfectant substance

(76) Inventors: Rene Challand, Geneve (CH); Alain Trachsel, Gland (CH); Josiane Trachsel, Gland (CH)

Correspondence Address: HENDERSON & STURM LLP, 1213 MIDLAND BUILDING, 206 SIXTH AVENUE, DES MOINES, IA, 50309-4076, US

(21) Appl. No.: 10/071,050

(22) Filed: Feb. 8, 2002

Related U.S. Application Data
(63) Continuation of application No. PCT/IB00/01063, filed on Jul. 31, 2000.

(30) Foreign Application Priority Data
Aug. 9, 1999 (EP) 99.810717.1

(51) Int. Cl. A61L 9/01

(52) U.S. Cl. 422/5

(57) ABSTRACT

A method and apparatus for dispersing a substance, in particular a fragrant or disinfectant substance, into a volume of air comprises a first step of circulating a volume of air inside a conduit and a second step of liberating the substance into the conduit. Circulation of the volume of air ensures ventilation for example of a vehicle, a restaurant, a cinema, or furthermore, a hospital. The substance liberated into the conduit is dispersed in the volume of air by forced convection and leads to a perfumed environment or to air containing an active disinfectant principle. A cylinder is positioned outside the conduit containing the substance to be dispersed and a compressed propellant gas, and the propellant gas is expanded so as to inject the substance into the conduit.

### CROSS REFERENCE TO RELATED APPLICATIONS

This application is a Continuation application of PCT/IB00/01063 filed Jul. 31, 2000, entitled Method and Device for Releasing a Fragrant of Disinfecting Substance, which claims priority of European Application No. 99.810717.1 filed Aug. 9, 1999, which are incorporated in their entirety herein.

### BACKGROUND OF THE INVENTION

1. Field of the Invention

This invention relates to a method and a device for liberating a substance, in particular a fragrant or disinfectant substance into a volume of air.

2. Description of Related Art

At the present time, many devices exist for liberating a fragrant substance which must be deposited in a volume of air itself where the substance is to be liberated. In these devices, for example incense sticks or apparatuses for heating natural essences, the substance is conveyed into the volume of air mainly by diffusion and natural convection. These methods of conveyance are relatively slow and do not enable the substance to be dispersed in a uniform manner in the volume of air. Moreover, adjustment of the quantity of substance emitted by the device is rudimentary and simply consists of putting the device into service or stopping it.

During use, an excessive or insufficient concentration of the substance is frequently arrived at, according to whether the space is too small or too large, compared with the quantity emitted by the device. It is unnecessary to recall that the quantity of perfume creating an agreeable sensation for a human being is usually of the order of a few parts per million (ppm). The same is true for disinfectant substances for which the quantity liberated must not be greater than a toxicity threshold.

In order to increase the uniformity of dispersion and to control more easily the quantity of substance emitted, it is preferred to circulate a volume of air in a conduit and to liberate the fragrant or disinfectant substance inside the conduit. Circulation of air in the conduit creates forced convection which produces a more uniform dispersion of the substance in the volume of air to be treated.

The fragrant or disinfectant substance is generally dissolved in a solvent with which it forms a liquid organic composition. The organic composition may also consist of the fragrant or disinfectant substance in the pure state. The liquid is contained in a cartridge. In order to disperse the substance in the volume of air, the cartridge is installed close to the conduit and liquid is forced with the aid of the compressor so as to be ejected in the form of fine droplets outside the cartridge into a filter acting as a diffuser positioned in the conduit, the fragrant substance being entrained by the air to be treated circulating in the conduit.

In this type of known method, it is found that control of the quantity of substance liberated in the volume of air has a large inertia due to the compressor. A delay exists between the command sent to the compressor and an actual change of the quantity of substance liberated into the volume of air. Liberation on demand or intermittently, so as to provide a fine dose of the quantity of the substance emitted, is not easily achieved with a compressor dedicated primarily to a permanent regime.

A method is known from U.S. Pat. Nos. 4,903,583 or 5,716,011 for dispersing a substance, in particular a fragrant or disinfectant substance, into a volume of air, in which method a volume of air is made to flow in a conduit and a fragrant or disinfectant substance is liberated inside the conduit by disposing outside the conduit a cylinder containing the substance to be dispersed and a compressed propellant gas and by expanding the propellant gas so as to inject the substance into the conduit.

The propellant gas contained in the cylinder under pressure expands with a negligible response time. In this way, the propellant gas immediately carries the fragrant or disinfectant substance to the injection region of the conduit. Rapid injection provides a fine dose of the substance liberated in the volume of air flowing in the conduit. If the substance to be liberated has to be changed or the method has to be reloaded, the cylinder is simply replaced without any operation in the conduit.

The method described in U.S. Pat. No. 4,903,583 more particularly employs a control unit for a fan enabling a flow of air to be established in the conduit and a means of starting and stopping the expansion of the propellant gas contained in the cylinder. The time for starting the fan and expanding the propellant gas is determined as a function of internal clocks in the control unit.

The method described in U.S. Pat. No. 5,716,011 more particularly employs a cylinder equipped with a valve and a flow meter maintaining a constant flow of emerging propellant gas so as to obtain a substantially constant concentration of substance in the regularly renewed volume of air of a room.

### BRIEF SUMMARY OF THE INVENTION

A method and apparatus for dispersing a substance, in particular a fragrant or disinfectant substance, into a volume of air comprises a first step of circulating a volume of air inside a conduit and a second step of liberating the substance into the conduit. Circulation of the volume of air ensures ventilation for example of a vehicle, a restaurant, a cinema, or furthermore, a hospital. The substance liberated into the conduit is dispersed in the volume of air by forced convection and leads to a perfumed environment or to air containing an active disinfectant principle.

A cylinder is positioned outside the conduit containing the substance to be dispersed and a compressed propellant gas, and the propellant gas is expanded so as to inject the substance into the conduit.

One of the objects of the invention is to treat a volume of air flowing in a conduit by dispersing a fragrant or disinfectant substance so that the quantity of substance emitted is easily controlled and an accurate dose of the substance is therefore provided in the volume of air.

To this end, the invention concerns a method, characterized in that injection of the substance is controlled as a function of an indicated value of the propellant gas pressure in the chamber and an indicated value of the flow rate of the air passing through the conduit. Indicated values of the propellant gas pressure and of the flow rate of the air passing through are used so as to obtain a concentration of the substance dispersed in the volume of air that is as linear as possible.

In an equally advantageous manner, injection of the propellant gas is interrupted when the air pressure from the conduit passes below a low pressure threshold. When the pressure in the conduit falls and crosses this low threshold, injection is interrupted for safety's sake so as to prevent an accumulation of propellant gas in the conduit.

The invention extends to a device for liberating a substance, in particular a fragrant or disinfectant substance, in a volume of air flowing in a conduit, comprising an injection pipe mounted inside the conduit and an external cylinder containing the substance to be dispersed together with a propellant gas under pressure, and a valve mounted on a communicating connection between the cylinder and the injection pipe, the propellant gas being expanded on opening the valve so as to convey a quantity of substance to be dispersed from the cylinder into the conduit through the injection pipe, characterized in that it includes a pressure sensor determining the pressure in the cylinder and a flow rate sensor determining the flow rate of the air passing through the conduit.

Advantageously, the valve is a solenoid valve controlled by computerized means such as a microcontroller or a microprocessor. This arrangement provides flexible management and automatic control of the quantity of substance dispersed. Provision is made to program the microcontroller so as to control the solenoid valve at a distance with the aid of a microcomputer.

Compared with a compressor forcing the liquid to be ejected out from the cartridge into the conduit, the cylinder containing liquid organic composition and propellant gas provides a reduction in weight and eliminates moments of immobilisation for inevitable purges, maintenance and repair of the compressor. Moreover, controlling the solenoid valve with the aid of a microcontroller or a microprocessor proves to be simpler than controlling a compressor.

### BRIEF DESCRIPTION OF THE DRAWINGS

Other features and advantages of the invention will become apparent on reading the detailed description of an embodiment illustrated by the drawings.

FIG. 1 is a schematic diagram of a device of the invention.

FIG. 2 shows a programming diagram of a microcontroller controlling a solenoid valve in a device of the invention.

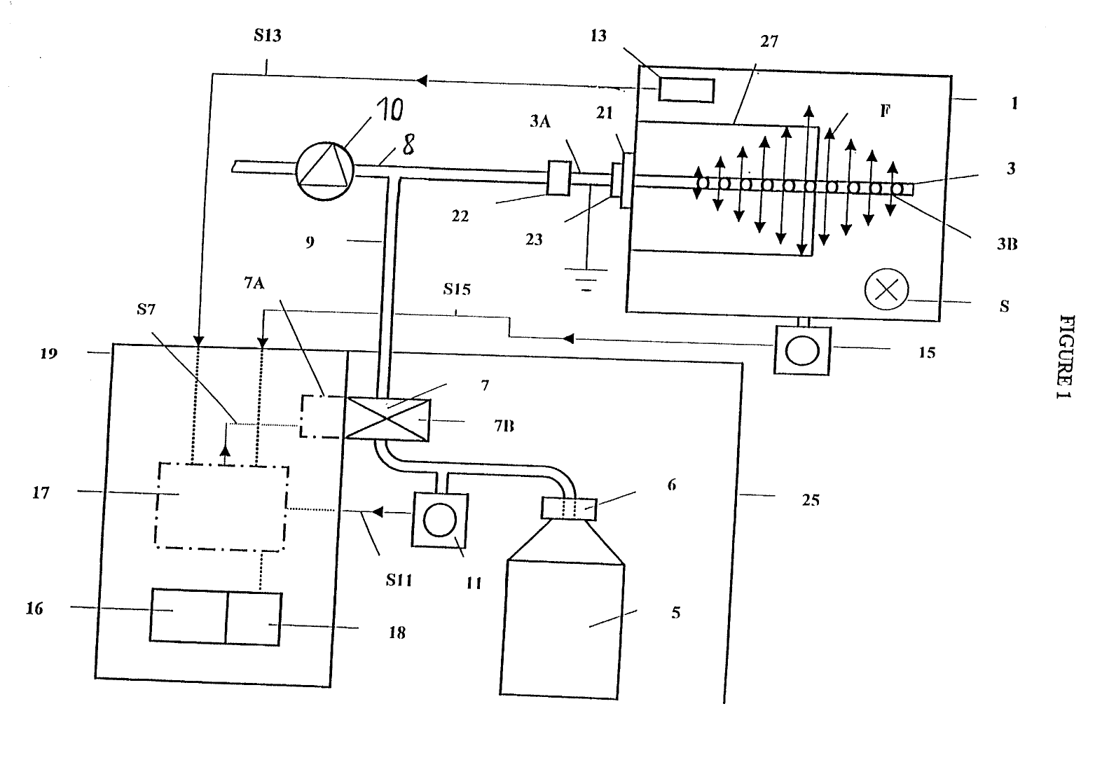

In FIG. 1, reference 1 denotes a ventilation conduit in which a volume of air circulates. Air circulation is produced perpendicular to the plane of FIG. 1 as indicated by the symbol S in a cross positioned inside the conduit. The device for liberating a fragrant or disinfecting substance into the conduit comprises an injection pipe 3 mounted inside the conduit 1, a cylinder 5 positioned outside the ventilation conduit 1 and a valve 7 positioned on a communicating connection 9 between the cylinder 5 and the injection pipe 3. The cylinder 5 contains the substance dissolved in a solvent with which it forms a liquid organic composition. The organic composition may also consist of the fragrant or disinfectant substance in the pure state.

On opening the valve 7, the propellant gas expands so as to inject the substance to be dispersed into the ventilation conduit 1 through the communicating connection 9 and the injection pipe 3. Injection of the substance into the conduit, symbolized in FIG. 1 by the arrows F, is carried out rapidly after opening the valve, due to the immediate expansion of the propellant gas.

According to the invention, the injection of the substance is controlled as a function of an indicated value of the pressure of the propellant gas in the cylinder and an indicated value of the flow rate of the air circulating in the conduit. In FIG. 1, the reference 11 denotes a pressure sensor mounted on the communicating connection 9 between the valve 7 and the cylinder 5, and the reference 13 denotes a flow rate sensor mounted on the ventilation conduit 1.

Values of the pressure of the propellant gas in the cylinder 5 and the flow rate of the air circulating in the conduit 1 are used in order to obtain as regular as possible injection of the substance. If the air flow rate in the conduit falls, the open time of the valve 7 is reduced. On the other hand, if the pressure of the propellant gas falls in the cylinder 5, the open time of the valve 7 is increased. This use of values given by the sensors 11 and 13 thus makes it possible to maintain a concentration of the substance dispersed in the volume of air that is as linear as possible.

According to an alternative embodiment, the value of the flow rate of the air circulating in the ventilation conduit is fixed at a nominal value, and a temperature sensor (not shown) is mounted in the conduit so as to correct this nominal value for variations in flow rate due to variations in temperature. A correction is in particular useful when the device is installed in a hot or temperate climatic zone, knowing that an increase in temperature reduces the mass air flow rate.

Even more preferably, the injection of propellant gas is interrupted when the pressure of the air in the conduit passes below a low pressure threshold. In FIG. 1, the reference 15 denotes a pressure sensor mounted in communication with the ventilation conduit 1. This arrangement makes it possible to prevent an accumulation of propellant gas in the conduit, which contributes to the safety of the device, in particular in the case where a flammable propellant gas is used. Injection is once again started when the pressure in the conduit becomes normal again.

In the embodiment chosen to illustrate the invention, the valve 7 is a solenoid valve controlled by a microcontroller or a microprocessor 17. As illustrated in FIG. 1, the microcontroller 17 and the electrical component 7A of the solenoid valve 7 are placed in a casing 19 which ensures separation of these elements from the mechanical part 7B of the solenoid 7, the cylinder 5 and the communicating connection 9. This arrangement increases the safety of the device as regards risks of explosion when flammable gases are used as the propellant gas or when the device itself is located in surroundings where there is a risk of explosion. Moreover, this arrangement prevents any electromagnetic disturbances in the area surrounding the device, in accordance with the standards in force.

The sensor 11 for the pressure of the propellant gas, the flow rate sensor 13 or the temperature sensor, and as appropriate, the pressure sensor 15 for the volume of gas circulating in the conduit, are connected to the microcontroller 17 to which signals S11, S13 and S15 are sent indicating the values detected. The microcontroller 17 possesses analogue-digital converters for processing these signals according to an algorithm prerecorded in the microcontroller or loaded into a microcomputer driving the microcontroller at a distance. The algorithm is used to calculate an open time of the solenoid valve.

This arrangement makes it possible to manage the injection of the substance into the conduit with a great degree of flexibility. Apart from providing regular injection as a function of the flow rate or the temperature of the volume of air circulating and the pressure of the propellant gas, or furthermore interrupting injection in the case where the low pressure threshold in the conduit is crossed, the microcontroller makes it possible to program the ejection of the substance for example on a timed basis.

Provision is made to process the indicated value of the pressure of the propellant gas in the cylinder so that the microcontroller displays a signal that the cylinder should be replaced when it is empty. It is possible for example to store the initial quantity of the liquid organic composition contained in the cylinder in the memory of the microcontroller, as well as the relationship for extrapolating the remaining quantity as a function of the reduction in pressure of the propellant gas.

Preferably, the microcontroller is programmed by a status table. This type of programming makes it possible to obtain fine control and a high level of safety in the programming. Programming the microcontroller uses initial values such as the quantity L of the liquid organic composition contained in the cylinder or nominal values, such as the flow rate Q of the air circulating in the ventilation conduit or the quantity C of substance to be dispersed in the volume of air considered. The algorithm for calculating the open time T of the solenoid valve is based on these initial and nominal values, on the variable numerical values resulting from the conversion of the signals emitted by the pressure sensors, the flow sensors or the temperature sensors, as well as on extrapolation or correction curves. If a new quantity of the substance to be dispersed in the volume of air is fixed, the microcontroller calculates the new open time of the solenoid valve as a function of this new quantity and the numerical values emitted by the sensors.

As can be seen in FIG. 1, the initial or nominal values are entered by means of a keyboard 18 on one front face of the casing 19 so as to be transmitted to the microcontroller 17. The different values used by the algorithm are displayed on a screen 16 positioned at the side of the keyboard 18 on the front face of the casing 19.

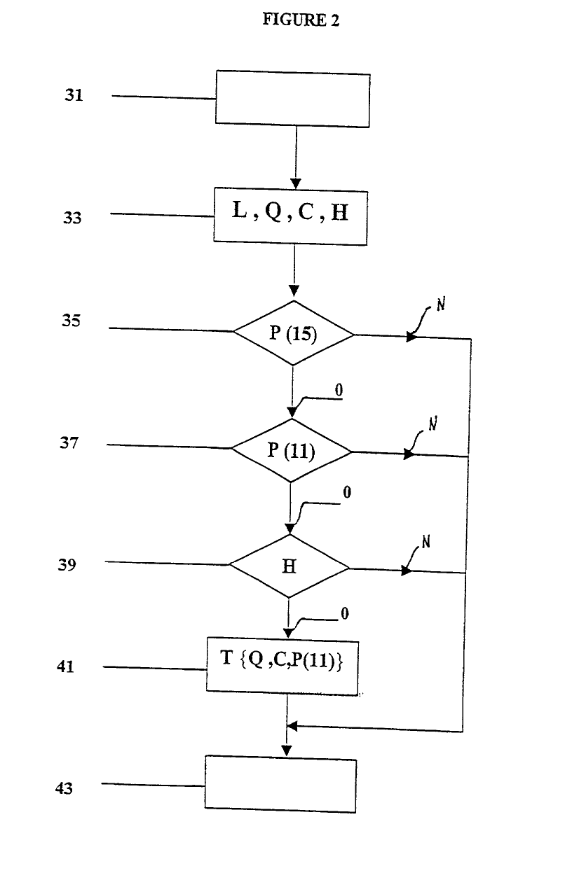

FIG. 2 illustrates an injection cycle performed by the microcontroller 17 for controlling the opening of the solenoid valve 7. References 31 and 43 denote the start and end of the injection cycle respectively. The reference 33 denotes the initialization of the microcontroller during which are fixed the initial quantity L of the liquid organic composition contained in the cylinder 5, the flow rate Q of air circulating in the ventilation conduit, the quantity C of substance to be dispersed in the volume of air, and the scheduled times H during which the substance is to be dispersed.

Reference 35 denotes a first test, during which the pressure P(15) of the air is checked with the aid of the pressure sensor 15 mounted on the ventilation conduit. If the pressure P(15) is below a low threshold indicating that the air is not circulating in the conduit, the microcontroller interrupts the injection cycle so as to prevent an accumulation of propellant gas in the unventilated conduit. Interrupting the cycle is symbolized by the arrow N in FIG. 2. The microcontroller sends a message to the screen 16 on the casing 19 signalling the absence of air circulation in the conduit. If on the contrary the pressure is greater than the low pressure threshold, the cycle is continued as indicated by the arrow O.

Reference 37 denotes a second test during which the pressure P(11) of the propellant gas in the cylinder 5 is checked with the aid of the pressure sensor 11. If the pressure P(11) is below a predetermined limit value indicating that the quantity of liquid organic composition contained in the cylinder is insufficient, the microcontroller shuts down the injection cycle as indicated by the arrow N and sends a message to the screen 16 of the casing 19 signalling that the contents of the cylinder are insufficient in liquid organic composition. If, on the contrary, the pressure P(11) is greater than the predetermined pressure, the cycle is continued as indicated by the arrow O.

Reference 39 denotes a third test, during which the scheduled time period H during which the substance is to be dispersed in the conduit is checked. If the time of the test is not included in the scheduled period, the microcontroller interrupts the cycle and sends a message to the screen signalling the waiting position of the device with respect to the scheduled period recorded.

In the opposite case, the microcontroller calculates an open time T for the solenoid valve according to the quantity C of substance to be dispersed in the volume of air, the flow rate Q of the air circulating in the ventilation conduit, and the pressure P(11) of the propellant gas in the cylinder. An opening command S7 is sent by the microcontroller 17 to the solenoid valve 7 as can be seen in FIG. 1. The microcontroller processes the signal S11 indicating the pressure of the propellant gas in the cylinder and the nominal value Q of the flow rate of the air circulating in the conduit or the signal S13 indicating this flow rate, so as to obtain a quantity C of substance dispersed that is as regular as possible. As indicated previously, if the air flow rate falls in the conduit, the open time of the valve is reduced. On the other hand, if the pressure of the propellant gas falls in the cylinder, the open time of the valve is increased.

It should be noted that the open time T of the solenoid valve takes account of the charge introduced through the communicating connection linking the valve to the ventilation conduit. At an equal pressure of propellant gas in the cylinder, the longer the communicating connection, for example, the longer the open time of the solenoid valve.

The quantity of substance propelled by the propellant gas during the open time of the valve is advantageously conveyed to the ventilation conduit by an auxiliary flow injected into the communicating connection downstream from the valve. As can be seen in FIG. 1, the auxiliary flow consists of an airflow created by the pump 10 discharging into the injection circuit 8 mounted as a bypass with respect to the communicating connection 9.

By means of this arrangement, the charge introduced through the communicating connection is reduced, which makes it possible to reduce the open time of the valve. The auxiliary flow also makes it possible to increase the speed at which the substance propelled by the propellant gas outside the cylinder is conveyed in the communicating connection. The auxiliary flow may also be used for rapidly purging the communicating connection during a change of cylinder. The quantity of substance to be liberated in the volume of air is advantageously calculated by the microcontroller or microprocessor as a function of a variable representing an initial quantity of pollutant material in the volume of air. The device described previously includes to this end a sensor connected to the microcontroller or to the microprocessor and designed so as to determine the quantity of pollutant material present in the volume of air. Preferably, this sensor is an electronic nose. The substance to be dispersed is a pure liquid organic composition or a fragrant or disinfectant principle contained in solution in a solvent, for example ethyl alcohol.

The propellant gas is a mixture of the butane, propane and isobutane type for applications where the volume of air is not exposed to sources of heat that could bring about ignition of the gas. In the case of a risk of ignition, nitrogen, carbon dioxide or air itself is used as the propellant gas. It should be noted that alkanes provide greater solubility of the organic substance in the air than nitrogen or carbon dioxide.

The injection pipe 3 is in the form of a metal or plastic tube 3A drilled with injection holes 3B. The tube 3A is connected at one end to the ventilation conduit 1 via a fixing plate 21 and a threaded connection 23 provided with a seal. The end of the tube opposite the communicating connection is stoppered.

The diameter of the injection holes 3B is preferably greater in the central region of the conduit than it is close to its walls. This arrangement encourages the distribution of the substance in the center of the conduit and contributes to greater uniformity of the substance in the volume of air. It should be noted that the injection pipe 3 is withdrawn from the conduit 1 independently of the cylinder S by means of the intermediate threaded connection 22 between the communicating connection 9 and the tube 3A. As can also be seen in FIG. 1, the injection pipe is bonded to earth so as to prevent any accumulation of electrical charges.

Provision may be made to use injection nozzles in the place of the injection pipe. Provision may also be made to use other systems, for example filters, for splitting up the organic composition into droplets or for vaporising it.

In the example illustrating the invention, the cylinder 5 is protected by a hood 25 secured to the casing 19. The latter is fixed to a wall or mounted directly on the ventilation conduit. According to the quantity of substance to be dispersed in the volume of air, cylinders of different capacities are used, typically of 100 cm³ to 1000 cm³. For a flow rate of the air circulating in the conduit having a value below 1000 m³ per hour, a single cylinder is sufficient for a day's use at a concentration of a few ppm of substance dispersed in the volume of air. For an air flow greater than 1000 m³ per hour, a minimum of two cylinders is provided and a means for automatically switching from one to the other when all the organic compound of one is injected, or for replacing one cylinder by another containing a different substance, for example a different perfume. As indicated previously, injection of the substance into the conduit by expansion of the propellant gas contained together with the organic composition in the cylinder under pressure, makes it possible to reduce the response time of the device to a minimum when a new substance is dispersed into the volume of air circulating in the conduit. The valve 7 is compatible with the substance to be dispersed. In particular it is resistant to corrosion.

Advantageously, provision is made to position a deflector 27 for disturbing the laminar flow of the volume of air in the ventilation conduit 1 and thus contributing to a more effective dispersion of the substance.

The communicating connection 9 is made of stainless steel or plastic material and is designed to resist excess pressure. It is fixed to the cylinder 5 by an end piece 6 also in stainless steel or in plastic material, making it possible, by virtue of an internal valve, to unscrew the cylinder without the substance dispersing into the ambient volume.

It should be noted that in this embodiment, the device has the following additional advantages: low weight, reduced overall size, low electricity consumption and minimum maintenance.

According to another embodiment of the invention (not shown) the device is installed on a vehicle and the battery of the vehicle is used as the electrical supply of the microcontroller and the solenoid valve. The volume of the cylinder is typically 100 cm³ and the propellant gas is preferably nitrogen or carbon dioxide. The dimensions of the injection pipe are adapted to the ventilation conduit of the vehicle inside which it is mounted. The concentration of the substance dispersed in the volume of air of the vehicle is adjusted according to the nominal value of the flow rate of air circulating in the ventilation conduit and of the pressure in the cylinder. A correction for the open time of the solenoid valve as a function of the temperature of the circulating air is easily achieved by a temperature sensor mounted in the ventilation conduit.

It should be noted that the fragrant or disinfectant character of the substance to be dispersed is not in itself a deciding factor for the invention. The latter applies to any substance that can be dispersed in air. The number of cylinders or injection pipes is also not a deciding factor.

Similarly, the ventilation conduit may be traversed by a fluid other than air without the method and device described previously being modified.

Finally, it should be noted that the invention is not limited to putting the volume of air into movement by circulation in a ventilation conduit. Other types of flow are possible for the volume of air. Provision may be made in particular to cause a volume of air, that is initially contained in a chamber under pressure, to flow through a conduit. The volume of air is injected into the conduit at the same time as the substance is injected by expansion of the propellant gas contained in the cylinder. The period during which the substance is injected is set as a function of the period during which the volume of air flows through the conduit, and the air flow rate. In a method of operation controlled by a computer, the microcontroller previously described is used to control the opening of the solenoid valve of the cylinder and at the same time the opening of a solenoid valve mounted on the chamber containing the volume of air under pressure.

This mode of flow is particularly well suited for treating small volumes of air on demand. As an example, a conduit is positioned emerging close to a video screen and a volume of air is injected into the conduit at the same time as quantity of the fragrant substance of which the perfume corresponds to the images displayed on the screen. In this way, an olfactory ambience is created around the television screen in association with the image seen on the screen.

1. A method for dispersing a substance, in particular a fragrant or disinfectant substance, in a volume of air, in which method a volume of air is made to flow inside a conduit (1) and a fragrant or disinfectant substance is liberated inside the conduit by positioning a cylinder (5) containing the substance to be dispersed and a compressed propellant gas outside the conduit (1), and by expanding the propellant gas so as to inject the substance inside the conduit, wherein injection of the substance is controlled as a function of an indicated value of the pressure (P11) of the propellant gas in the cylinder and of an indicated value of the flow rate (Q) of the air passing through the conduit.

2. A method according to claim 1, wherein injection of the propellant gas is discontinued when the air pressure in the conduit (P15) passes below a low pressure threshold.

3. A method according to claim 1, wherein the substance propelled by the propellant gas is conveyed to the conduit by the injection of an auxiliary flow.

4. A method according to claim 1, wherein the substance is injected by opening a solenoid valve (7) controlled by a microcontroller or a microprocessor (17) programmed for:
a) initialising (33) a variable representing the quantity (L) of substance contained in the cylinder, a variable representing the throughput (Q) of air flowing in the conduit, and a variable representing the quantity (C) of substance to be dispersed in the volume of air,
b) checking, in the form of a first test (35), the air pressure (P15) in the conduit,
c) checking, in the form of a second test (37), the pressure (P11) of the propellant gas in the cylinder, and
d) opening the solenoid valve (7) during an open time (T), calculated as a function of pressure (P11), of the propellant gas, of the flow rate (Q) of the air passing through the conduit, and of the quantity (C) of substance to be liberated in the volume of air.

5. A method according to claim 2, wherein the substance is injected by opening a solenoid valve (7) controlled by a microcontroller or a microprocessor (17) programmed for:
a) initialising (33) a variable representing the quantity (L) of substance contained in the cylinder, a variable representing the throughput (Q) of air flowing in the conduit, and a variable representing the quantity (C) of substance to be dispersed in the volume of air,
b) checking, in the form of a first test (35), the air pressure (P15) in the conduit,
c) checking, in the form of a second test (37), the pressure (P11) of the propellant gas in the cylinder, and
d) opening the solenoid valve (7) during an open time (T), calculated as a function of pressure (P11), of the propellant gas, of the flow rate (Q) of the air passing through the conduit, and of the quantity (C) of substance to be liberated in the volume of air.

6. A method according to claim 3 wherein the quantity of substance (C) to be liberated in the volume of air is calculated by the microcontroller or microprocessor as a function of a variable representing an initial quantity of pollutant material in the volume of air.

7. A method according to claim 1, wherein the volume of air is made to flow inside the conduit (1) by injection from a compressed chamber containing air under pressure, or by circulation.

8. A method according to claim 2, wherein the volume of air is made to flow inside the conduit (1) by injection from a compressed chamber containing air under pressure, or by circulation.

9. A method according to claim 3, wherein the volume of air is made to flow inside the conduit (1) by injection from a compressed chamber containing air under pressure, or by circulation.

10. A method according to claim 4, wherein the volume of air is made to flow inside the conduit (1) by injection from a compressed chamber containing air under pressure, or by circulation.

11. A method according to claim 5, wherein the volume of air is made to flow inside the conduit (1) by injection from a compressed chamber containing air under pressure, or by circulation.

12. A method according to claim 6, wherein the volume of air is made to flow inside the conduit (1) by injection from a compressed chamber containing air under pressure, or by circulation.

13. A device for liberating a substance, in particular a fragrant or disinfectant substance, into a volume of air flowing in a conduit (1) comprising an injection pipe (3) mounted inside the conduit, an external cylinder (5) containing the substance to be dispersed together with a propellant gas under pressure, and a valve (7) mounted on a communicating connection (9) between the cylinder and the injection pipe, the propellant gas expanding on opening the valve so as to convey a quantity of the substance to be dispersed from the cylinder into the conduit via the injection pipe, it additionally including a pressure sensor (11) determining the pressure in the cylinder and a flow rate sensor (13) determining the flow rate of the air passing through the conduit.

14. A device according to claim 13, and further comprising a temperature sensor determining the temperature of the air flowing in the conduit.

15. A device according to claim 13, and further comprising a pressure sensor (15) determining the pressure in the conduit.

16. A device according to claim 13, and further comprising a circuit for injecting an auxiliary flow mounted as a bypass in relation to the communicating connection.

17. A device according to claim 13, wherein the valve (7) is a solenoid valve controlled by a microcontroller or a microprocessor (17).

18. A device according to claim 17, wherein the microcontroller (17) is linked to a sensor (11) indicating the pressure of the propellant gas in the cylinder (5) and a sensor (13) indicating the flow rate of the air passing through the conduit (1).

19. A device according to claim 18, and further comprising a sensor determining a quantity of pollutant matter present in the volume of air and linked to the microcontroller or microprocessor.

20. A device according to claim 13, wherein the injection pipe (3) is provided with holes (3B) of which the diameter is greater when the hole is positioned in a part of the pipe that is more central with respect to the conduit (1).

21. A method of using the device of claim 17, wherein the device is fitted on board a vehicle by installing the injection pipe (3) in a ventilation conduit (1) of the vehicle, the substance is injected into the conduit by expanding a propellant gas, and the solenoid valve (7) and the microcontroller or microprocessor (17) are supplied with the aid of a self-contained battery fitted on board the vehicle.
